(19) United States
(12) Patent Application Publication
(10) Pub. No.: US 2024/0158849 A1
(43) Pub. Date: May 16, 2024

(54) COMPOSITIONS AND METHODS FOR SEQUENCING USING POLYMERS WITH METAL-COATED REGIONS AND EXPOSED REGIONS

(71) Applicant: Illumina, Inc., San Diego, CA (US)

(72) Inventors: Rico Otto, San Diego, CA (US); Hayden Black, San Diego, CA (US)

(73) Assignee: Illumina, Inc., San Diego, CA (US)

(21) Appl. No.: 18/552,524

(22) PCT Filed: Feb. 8, 2022
(86) PCT No.: PCT/US2022/015643

§ 371 Date: Sep. 26, 2023

Related U.S. Application Data
(60) Provisional application No. 63/167,817, filed on Mar. 30, 2021.

(51) Int. Cl. C12Q 1/6869; G01N 27/327

(52) U.S. Cl. CPC C12Q 1/6869; G01N 27/3275

(57) ABSTRACT

Provided herein are compositions and methods for sequencing using metal-coated polymers. In some examples, a bridge spans a space between first and second electrodes and includes a polymer chain having a first metal-coated region contacting the first electrode, a second metal-coated region contacting the second electrode, and an exposed region located between the first and second regions. The composition includes first and second polynucleotides; a plurality of nucleotides, each nucleotide coupled to a corresponding label; and a polymerase to add nucleotides of the plurality of nucleotides to the first polynucleotide using at least a sequence of the second polynucleotide. The composition includes detection circuitry to detect a sequence in which the polymerase adds the nucleotides to the first polynucleotide using at least changes in an electrical signal through the bridge, the changes being responsive to contact between the labels corresponding to those nucleotides and the exposed region.

### CROSS-REFERENCE TO RELATED APPLICATION

This application claims to the benefit of U.S. Provisional Patent Application No. 63/167,817, filed on Mar. 30, 2021 and entitled “COMPOSITIONS AND METHODS FOR SEQUENCING USING POLYMERS WITH METAL-COATED REGIONS AND EXPOSED REGIONS”, the entire contents of which are incorporated by reference herein.

### FIELD

This application relates to compositions and methods for sequencing polynucleotides using metal coated polymers.

### BACKGROUND

A significant amount of academic and corporate time and energy has been invested into sequencing polynucleotides, such as DNA. Some sequencing systems use “sequencing by synthesis” (SBS) technology and fluorescence-based detection. However, fluorescence-based detection may require optical components such as excitation light sources, imaging devices, and the like, which may be complex, time-consuming to operate, and costly.

### SUMMARY

Examples provided herein are related to sequencing using metal-coated polymers. Compositions and methods for performing such sequencing are disclosed.

In some examples provided herein is a composition. The composition may include first and second electrodes separated from one another by a space. The composition may include a bridge spanning the space between the first and second electrodes. The bridge may include a polymer chain having a first metal-coated region contacting the first electrode, a second metal-coated region contacting the second electrode, and an exposed region located between the first and second regions. The composition may include first and second polynucleotides, a plurality of nucleotides, each nucleotide coupled to a corresponding label, and a polymerase to add nucleotides of the plurality of nucleotides to the first polynucleotide using at least a sequence of the second polynucleotide. The composition may include detection circuitry to detect a sequence in which the polymerase adds the nucleotides to the first polynucleotide using at least changes in an electrical signal through the bridge. The changes may be responsive to contact between the labels corresponding to those nucleotides and the exposed region.

In some examples, the polymer chain includes a polynucleotide. In some examples, the polynucleotide includes DNA. In some examples, the polynucleotide is single-stranded. In some examples, the polynucleotide is double-stranded.

In some examples, the first and second metal-coated regions include metal nanoparticles coupled to the polymer chain.

In some examples, the first and second metal-coated regions include metal plated on the polymer chain.

In some examples, the metal is selected from the group consisting of: silver, platinum, palladium, gold, copper, nickel, cobalt, zinc, and rhodium.

In some examples provided herein is a method. The method may include adding, using a polymerase, nucleotides to a first polynucleotide using at least a sequence of a second polynucleotide. The method may include contacting, using labels respectively coupled to the nucleotides, an exposed region of a bridge spanning a space between first and second electrodes. The bridge may include a polymer chain having a first metal-coated region contacting the first electrode, a second metal-coated region contacting the second electrode, and the exposed region located between the first and second metal-coated regions. The method may include detecting a sequence in which the polymerase adds the nucleotides to the first polynucleotide using at least changes in electrical signal through the bridge that are responsive to respective contact between the labels corresponding to those nucleotides and the exposed region.

In some examples, the polymer chain includes a polynucleotide. In some examples, the polynucleotide includes DNA. In some examples, the polynucleotide is single-stranded. In some examples, the polynucleotide is double-stranded.

In some the first and second metal-coated regions include metal nanoparticles coupled to the polymer chain.

In some examples, the first and second metal-coated regions include metal plated on the polymer chain.

In some examples, the metal is selected from the group consisting of: silver, platinum, palladium, gold, copper, nickel, cobalt, zinc, and rhodium.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate an example composition for sequencing including a bridge that includes a polymer with metal-coated regions and an exposed region, and nucleotide labels that contact the exposed region to alter an electrical characteristic of the bridge.

FIGS. 2A-2C schematically illustrate examples of nucleotides with labels for use with a bridge that includes a polymer with metal-coated regions and an exposed region.

FIG. 3 schematically illustrates an example composition for sequencing including a bridge that includes a polymer with metal-coated regions and an exposed region.

FIG. 4 illustrates an example flow of operations in a method for sequencing using a bridge that includes a polymer with metal-coated regions and an exposed region, and nucleotide labels that contact the exposed region to alter an electrical characteristic of the bridge.

FIGS. 5A-5D schematically illustrate an example flow of operations in a method for making a bridge that includes a polymer with metal-coated regions and an exposed region.

FIGS. 6A-6B schematically illustrate another example flow of operations in a method for making a bridge that includes a polymer with metal-coated regions and an exposed region.

FIGS. 7A-7B schematically illustrate another example flow of operations in a method for making a bridge that includes a polymer with metal-coated regions and an exposed region.

FIGS. 8A-8C schematically illustrate another example flow of operations in a method for making a bridge that includes a polymer with metal-coated regions and an exposed region.

FIGS. 9A-9B are atomic force microscopy (AFM) images of individual DNA duplexes combed across a space between electrodes, according to one example.

FIG. 10 is an AFM image of DNA after seeding with silver ions and reducing to silver nanoparticles, according to one example.

FIGS. 11A-11C are scanning electron microscopy (SEM) images of metalized DNA that has undergone seeding with silver clusters and then electroless plating with gold, according to one example.

FIGS. 12A-12B are SEM images of DNA that was combed across a space between electrodes, seeded with silver clusters, and then electroless plated with gold, according to one example.

### DETAILED DESCRIPTION

Examples provided herein are related to sequencing using sequencing using metal-coated polymers. Compositions and methods for performing such sequencing are disclosed.

More specifically, the present compositions and methods suitably may have the benefits of being used to sequence polynucleotides in a manner that is robust, reproducible, sensitive, accurate, works in real time, detects single molecules, and has high throughput. For example, the present compositions can include first and second electrodes and a bridge that spans the space between the electrodes. The bridge can include a partially metal coated-polymer, e.g., can include a polymer chain via which electrical current may flow from one electrode to another through the bridge, and that has first and second metal-coated regions separated from one another by an exposed region. The first metal-coated region may contact the first electrode and the second metal-coated region may contact the second electrode, to form an ohmic contact. Since charge can easily flow in both directions between the electrode and the metal-coated region, the ohmic contact allows for injecting charge into the bridge with reduced risk, or substantially no risk, of current-induced damage to the polymer chain itself, or the junction between the electrode and the polymer chain, which otherwise may lead to detachment of the bridge from the electrodes. The metal-metal contact between the electrode and the metal coated polymer chain therefore has a higher stability than a contact between the electrode and an uncoated polymer chain. The electrical current that flows through the bridge may be limited by the total electrical conductivity of the bridge, which primarily may be dominated by the electrical conductivity of the exposed region. Labels, which may be coupled to respective nucleotides, may alter one or more electrical characteristics of the bridge, for example the electrical conductivity or electrical impedance of the bridge, and using at least such alteration the respective nucleotide may be identified. For example, an electrical signal through the bridge may change responsive to contact between the labels corresponding to those nucleotides and the exposed region.

First, some terms used herein will be briefly explained. Then, some example compositions and example methods for sequencing polynucleotides will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially”, “approximately”, and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “electrode” is intended to mean a solid structure that conducts electricity. Electrodes may include any suitable electrically conductive material including metals such as gold, palladium, or platinum, or carbon based electrodes such as graphene or carbon nanotubes, or combinations thereof.

As used herein, the term “bridge” is intended to mean a structure that extends between, and couples to, two other structures. A bridge may span a space between other structures, such as between two electrodes. Not all elements of a bridge need to be directly coupled to both structures. For example, in a bridge that includes first and second polymer chains associated with one another and spanning the space between two electrodes, at least one end of one of the polymer chains is coupled to one of the electrodes, and at least one end of one of the polymer chains is coupled to the other electrode. However, both polymer chains need not be coupled to both of the electrodes, and indeed one of the polymer chains need not be coupled to either of the electrodes. A bridge may include multiple components which are coupled to one another in such a manner as to extend between, and collectively connect to, other structures. A bridge may be coupled to another structure, such as an electrode, via a chemical bond, e.g., via a covalent bond, hydrogen bond, ionic bond, dipole-dipole bond, London dispersion forces, metallic bond, or any suitable combination thereof. For example, a metal may couple a polymer chain of a bridge to an electrode via a metallic bond. By “metallic bond” it is meant a bond via which electrons may be shared freely among a structure of positively charged ions (cations).

As used herein, “metal” is intended to mean a material having a very high electrical and thermal conductivity and that is solid at room temperature. Nonlimiting examples of metals include silver (Ag), platinum (Pt), palladium (Pd), gold (Au), copper (Cu), nickel (Ni), cobalt (Co), zinc (Zn), and rhodium (Rh).

As used herein, a “polymer” refers to a molecule including a chain of many subunits, that may be referred to as monomers, that are coupled to one another. The subunits may repeat, or may differ from one another. Some polymers are “conjugated,” which is intended to mean that the monomers constituting such polymers are connected through sp² hybridized atomic centers to provide a delocalized set of molecular orbitals via which electrons may flow along the length of the polymer. Nonlimiting examples of conjugated polymers, and their constituent monomers, are provided further below. Polymers and their subunits can be biological or synthetic, and may be of any of the possible topological structures including linear, branched, star-shaped. The term “polymer” also includes more complicated polymer structures such as ladder polymers, rotaxanes, and catenanes.

Example biological polymers that suitably can be included within a bridge or a label include polynucleotides (made from nucleotide subunits), polypeptides (made from amino acid subunits), polysaccharides, polynucleotide analogs, and polypeptide analogs. Example polynucleotides and polynucleotide analogs suitable for use in a bridge or a label include DNA, enantiomeric DNA, RNA, PNA (peptide-nucleic acid), morpholinos, and LNA (locked nucleic acid). In one nonlimiting example, the polynucleotide may be in the form of DNA origami. Polymers may include spacer subunits, derived from phosphoramidites, which may be coupled to polynucleotides but which lack nucleobases, such as commercially available from Glen Research (Sterling, VA), for example spacer phosphoramidite 18 (18-O-Dimethoxytritylhexaethyleneglycol,1-[(2-cyanoethyl)-(N,N-diisopropyl)]-phosphoramidite). Polymers may include oligonucleotides with modified bases containing aromatic units that aid in electrical conductivity. Example synthetic polypeptides can include all natural amino acids, such as charged amino acids, hydrophilic, hydrophobic, and neutral amino acid residues. Example synthetic polymers that suitably can be included within a bridge or label include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON® (tetrafluoroethylene), thermoplastic polyurethanes, polyaldehydes, polyolefins, poly(ethylene oxides), poly(ω-alkenoic acid esters), poly(alkyl methacrylates), and other polymeric chemical and biological linkers such as described in Hermanson, Bioconjugate Techniques, third edition, Academic Press, London (2013).

“DNA origami” is intended to mean DNA with an intended tertiary structure. DNA origami may be constructed by mixing a single long DNA molecule, which may be referred to as a “template,” with short complementary sequences which may be called “staples.” Each staple may bind to specific regions within the long DNA molecule and pull the long DNA molecule into a desired shape. Each staple may have a unique sequence and may end up in a well-defined location in the final tertiary structure. Because every staple may be individually functionalized independently from any functionalization of other staples, this allows for exact placement of specific functional elements on the tertiary structure, such as a functional element that may be used to couple a polymerase, or a functional element that may be used to bond to an electrode. Example functional elements that may be included in or attached to one or more staples include, but are not limited to, nanoparticles, enzymes, chemical linkers, molecular wires such as carbon nanotubes, peptides, or other DNA origamis or DNA sequences. Relatively large DNA origami structures may be formed from multiple, smaller DNA origami structures.

As used herein, DNA with “tertiary structure” is intended to mean that the DNA is folded into a three-dimensional tertiary structure having internal cross-linking holding the folds in place. In comparison, DNA that has a primary structure (e.g., a particular sequence of nucleotides linked together) and a secondary structure (e.g., local structure) but no internal cross-linking holding folds into place would not be considered to have a tertiary structure as the term is used herein. For example, a double-stranded polynucleotide (e.g., dsDNA), a single-stranded polynucleotide (e.g., ssDNA), or a partially double-stranded (e.g., part dsDNA and part ssDNA) structure may be folded and cross-linked into a tertiary structure.

As used herein, a region of a polymer that is “metal-coated” is intended to mean that at least a portion of an outer surface of the polymer in that region is directly or indirectly coupled to a metal such that the metal, rather than the polymer, conducts a majority of the electrical current through that region. For example, the metal may surround, or substantially surround, the entire outer surface of the polymer in that region.

As used herein, a region of a polymer that is “exposed” is intended to mean that at least a portion of an outer surface of the polymer in that region is not directly or indirectly coupled to a metal or other material such that another element, such as a label, may directly contact the polymer in that region. For example, the entire, or substantially the entire, outer surface of the polymer in that region may be devoid of a metal.

As used herein, “hybridize” is intended to mean noncovalently associating a first polymer to a second polymer along the lengths of those polymers. For instance, two DNA polynucleotide strands may associate through complementary base pairing. The strength of the association between the first and second polymers increases with the complementarity between the sequences of monomer units within those polymers. For example, the strength of the association between a first polynucleotide and a second polynucleotide increases with the complementarity between the sequences of nucleotides within those polynucleotides.

As used herein, the term “nucleotide” is intended to mean a molecule that includes a sugar and at least one phosphate group, and in some examples also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dG1VIP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide can be a single stranded sequence of nucleotides, such as RNA or single stranded DNA, a double stranded sequence of nucleotides, such as double stranded DNA, or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are example examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded polynucleotide template, and can sequentially add nucleotides to the growing primer to form a polynucleotide having a sequence that is complementary to that of the template.

As used herein, the term “primer” is defined as a polynucleotide to which nucleotides are added via a free 3′ OH group. A primer may have a 3′ block preventing polymerization until the block is removed. A primer can also have a modification at the 5′ terminus to allow a coupling reaction or to couple the primer to another moiety. The primer length can be any number of bases long and can include a variety of non-natural nucleotides.

As used herein, the term “label” is intended to mean a structure that couples to a bridge in such a manner as to cause a change in an electrical characteristic of the bridge, such as electrical impedance or electrical conductivity, and based upon which change the nucleotide may be identified. For example, a label may interact with a polymer chain within such a bridge, and the interaction may cause an electrical conductivity or electrical impedance change of the bridge. For example, a label may hybridize to a polymer chain within bridge, or may intercalate between polymer chains within such a bridge, and the hybridization or intercalation may cause the electrical conductivity or electrical impedance change of the bridge. However, it should be appreciated that a label may alter any suitable electrical characteristic of a polymer chain within a bridge. In examples provided herein, labels can be coupled to nucleotides.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates can include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates can include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can comprise a single material or a plurality of different materials. Substrates can be composites or laminates. In some examples, the substrate comprises an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may comprise posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

### Example Compositions and Methods for Sequencing Polynucleotides

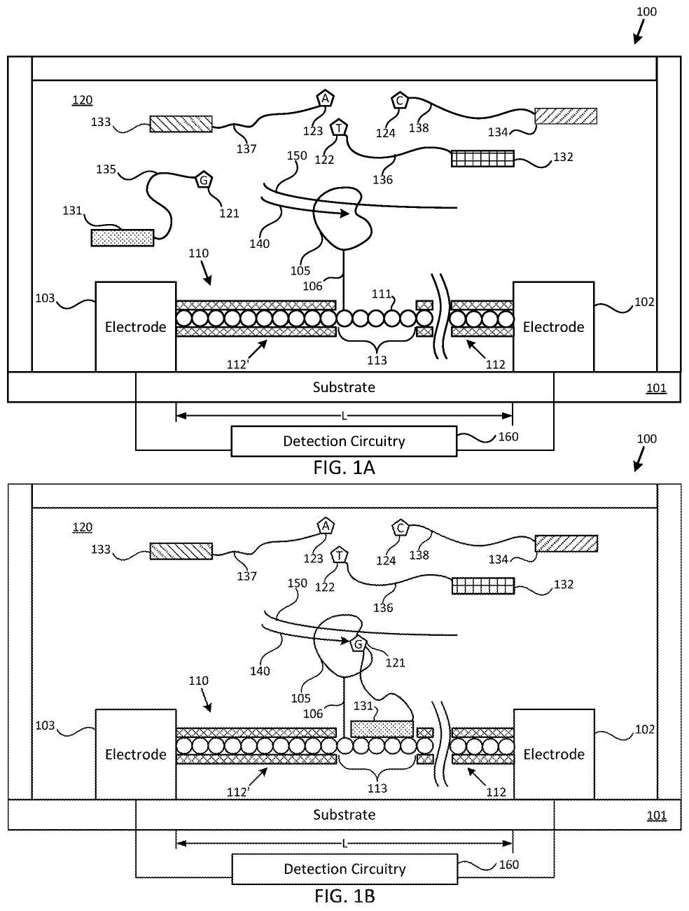

FIGS. 1A-1B schematically illustrate an example composition for sequencing including a bridge that includes a polymer with metal-coated regions and an exposed region, and nucleotide labels that contact the exposed region to alter an electrical characteristic of the bridge.

Referring now to FIG. 1A, composition 100 includes substrate 101, first electrode 102, second electrode 103, polymerase 105, bridge 110, nucleotides 121, 122, 123, and 124, labels 131, 132, 133, and 134 respectively coupled to those nucleotides, first polynucleotide 140, second polynucleotide 150, and detection circuitry 160. Polymerase 105 is in proximity of bridge 110, and in some examples may be coupled to bridge 110 via linker 106 in a manner such as known in the art. Such linker chemistries include maleimide chemistry to reactive thiols on cysteine residues, NHS ester chemistry to reactive amines on lysine residues, biotin-Streptavidin, and Spytag-SpyCatcher, for example. In the example illustrated in FIGS. 1A-1B, components of composition 100 may be enclosed within a flow cell filled with fluid 120 in which nucleotides 121, 122, 123, and 124 (with associated labels), polynucleotides 140, 150, and suitable reagents may be carried.

Substrate 101 may support first electrode 102 and second electrode 103. First electrode 102 and second electrode 103 may be separated from one another by a space, e.g., a space of length L as indicated in FIG. 1A. The value of L may be, in some examples, from about 1 nm to about 1 μm, e.g., from about 3 nm to about 1 μm, e.g., from about 3 nm to about 100 nm, e.g., from about 3 nm to about 50 nm, or e.g., from about 10 nm to about 1 μm, e.g., from about 100 nm to about 1 μm, e.g., from about 100 nm to about 500 nm. First electrode 102 and second electrode 103 may have any suitable shape and arrangement, and are not limited to the approximately rectangular shape suggested in FIG. 1A. The sidewalls of first electrode 102 and second electrode 103 illustrated in FIG. 1A may be, but need not necessarily be, vertical or parallel to one another, and need not necessarily meet the top surfaces of such electrodes at a right angle. For example, first electrode 102 and second electrode 103 may be irregularly shaped, may be curved, or include any suitable number of obtuse or acute angles. In some examples, first electrode 102 and second electrode 103 may be arranged vertically relative to one another. The value L may refer to the spacing between the closest points of first electrode 102 and second electrode 103 to one another.

Bridge 110 may span the space between first electrode 102 and second electrode 103, and may include polymer chain 111 (the circles within the respective polymer chains being intended to suggest monomer units that are coupled to one another along the lengths of the polymer chains). Polymer chain 111 may include a single polymer chain as is suggested in FIG. 1A (e.g., a single-stranded polynucleotide, such as ssDNA), or may include multiple polymer chains, e.g., first and second polymer chains with sequences that are complementary to one another (e.g., first and second polynucleotides, such as dsDNA). Polymer chain 111 may have length that is approximately the same as length L of the space between first electrode 102 and second electrode 103 or otherwise permits polymer chain 111 to span the space between first electrode 102 and second electrode 103, e.g., such that polymer chain 111 may be coupled directly to each of first electrode 102 and second electrode 103 (e.g., via respective bonds). It should be understood that in some configurations, polymer chain 111 may not necessarily coupled directly to one or both of first electrode 102 and second electrode 103. Instead, polymer chain 111 may be directly coupled to one or more other structures that respectively are coupled, directly or indirectly, to one or both of first electrode 102 and second electrode 103.

For example, polymer chain 111 of bridge 110 illustrated in FIG. 1A includes first metal-coated region 112 coupled to first electrode 102, second metal-coated region 112′ coupled to second electrode 103, and exposed region 113 disposed between the first and second metal-coated regions. The metal of first metal-coated region 112 may be coupled first electrode 102 via a metallic bond, and the metal of second metal-coated region 112′ may be coupled to second electrode 103 via a metallic bond, thereby coupling polymer chain 111 to the first and second electrodes. Metal-coated regions 112, 112′ may have any suitable diameter, e.g., a diameter between about 1 nm and about 200 nm, or between about 5 nm and about 200 nm, or between about 10 nm and about 100 nm, or between about 20 nm and about 50 nm. Exposed region 113 may have any suitable length, e.g., a length between about 1 nm and about 200 nm, or between about 5 nm and about 200 nm, or between about 10 nm and about 100 nm, or between about 20 nm and about 50 nm. Metal coated regions 112, 112′ may, but need not necessarily, have the same length as one another. Together, metal coated regions 112, 112′ and exposed region 113 may form substantially the entire length of bridge 110 in some examples. Metal-coated regions 112, 112′ each may include any suitable metal or metals, such as silver, platinum, palladium, gold, copper, nickel, cobalt, zinc, or rhodium, or any suitable combination thereof. The metal(s) of metal-coated regions 112, 112′ may be the same as the material of electrodes 102, 103, or may be different than the material of electrodes 102, 103. The electrical conductivity of metal-coated regions 112, 112′ may be relatively high as compared to the electrical conductivity of exposed region 113, where the electrical conductivity may express the material's conductance per unit area and length. For example, electrical conductance of metal-coated regions 112, 112′ may be in the range of about 1×10⁻⁵ to 1×10⁻⁴ S/cm.

As explained in greater detail herein, labels 131, 132, 133, and 134 respectively may alter an electrical characteristic of polymer chain 111 in such a manner as to modulate the electrical conductivity or impedance of bridge 110, based upon which modulation the identity of the corresponding nucleotides 121, 122, 123, and 124 may be determined. For example, as explained in greater detail with reference to FIG. 1B, labels 131, 132, 133, and 134 respectively may contact exposed region 113 in such a manner as to modulate the electrical conductivity or impedance of bridge 110, based upon which modulation the identity of the corresponding nucleotides 121, 122, 123, and 124 may be determined. In some examples, the labels may modulate a noise profile of polymer chain 111 in such a manner that the identity of the corresponding nucleotides may be determined.

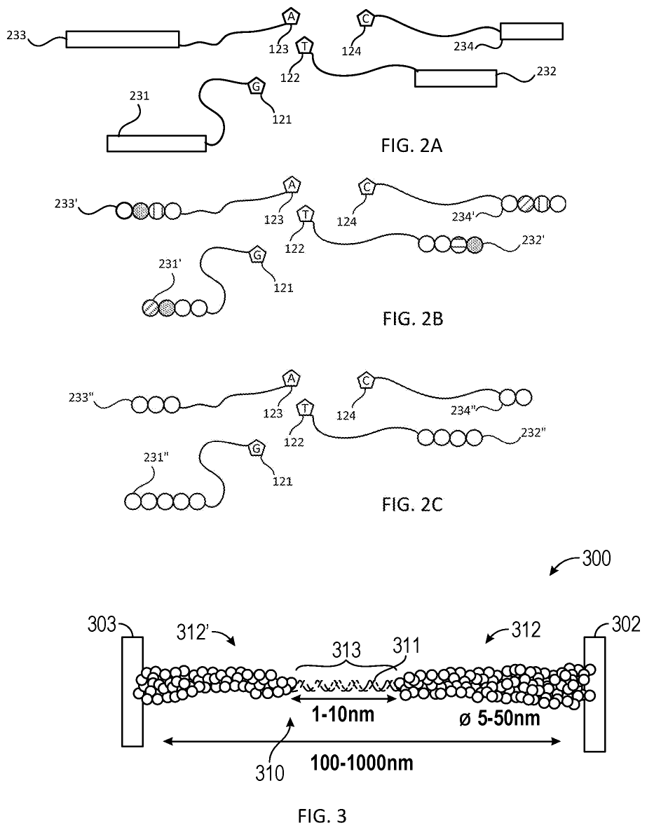

Composition 100 illustrated in FIG. 1A may include any suitable number of nucleotides coupled to corresponding labels, e.g., one or more nucleotides, two or more nucleotides, three or more nucleotides, or four nucleotides. For example, nucleotide 121 (illustratively, G) may be coupled to corresponding label 131, in some examples via linker 135. Nucleotide 122 (illustratively, T) may be coupled to corresponding label 132, in some examples via linker 136. Nucleotide 123 (illustratively, A) may be coupled to corresponding label 133, in some examples via linker 137. Nucleotide 124 (illustratively, C) may be coupled to corresponding label 134, in some examples via linker 138. The couplings between nucleotides and labels, in some examples via linkers which may include the same or different polymer as the labels, may be provided using any suitable methods known in the art, such as n-hydroxysuccinimide (NHS) ester chemistry or click chemistry. Labels 131, 132, 133, and 134 may differ from one another in at least one respect, as suggested by the different fill patterns in FIG. 1A. For example, as described below with reference to FIGS. 2A and 2C, labels 131, 132, 133, and 134 in some examples may have different lengths than one another. As another alternative, labels 131, 132, 133, and 134 in some examples may include the same type of polymer as one another, but may differ from one another in at least one respect, e.g., may have different sequences of monomer units than one another such as in the specific example described with reference to FIG. 2B. In some examples, labels 131, 132, 133, and 134 may include the same type of polymer as in exposed region 113, and in some examples may include the same type of polymer as in the remainder of one or both of polymer chains 111, 112. In a manner such as described in greater detail with reference to FIG. 1B, the particular characteristics of labels 131, 132, 133, and 134 may be respectively selected so as to facilitate generation of distinguishable electrical signals, such as currents or voltages, through bridge 110 when those labels respectively contact exposed region 113. The labels may, but need not necessarily, alter the same electrical characteristic as one another.

Composition 100 illustrated in FIG. 1A includes first polynucleotide 140 and second polynucleotide 150, and polymerase 105 that may add nucleotides of the plurality of nucleotides 121, 122, 123, and 124 to first polynucleotide 140 using at least a sequence of second polynucleotide 150. The labels 131, 132, 133, and 134 corresponding to those nucleotides respectively may alter an electrical characteristic of bridge 110, e.g., may contact exposed region 113 in a manner such as described in greater detail below with reference to FIG. 1B. Detection circuitry 160 may detect a sequence in which polymerase 105 respectively adds the nucleotides 121, 122, 123, and 124 (not necessarily in that order) to first polynucleotide 140 using at least changes in a current through or impedance of bridge 110, the changes being responsive to the contact between exposed region 113 and the labels 131, 132, 133, and 134 corresponding to those nucleotides. For example, detection circuitry 160 may apply a voltage across first electrode 102 and second electrode 103, and may detect any current that flows through bridge 110 responsive to such voltage.

At the particular time illustrated in FIG. 1A, none of labels 131, 132, 133, and 134 is in contact with bridge 110, and so a relatively high current may flow through bridge 110. Although nucleotides 121, 122, 123, 124 may diffuse freely through fluid 120 and respective labels 131, 132, 133, 134 may briefly contact bridge 110 as a result of such diffusion, the labels may relatively rapidly interact with the bridge and so any resulting changes to the electrical conductivity or impedance of bridge 110 are expected to be so short as either to be undetectable, or to be clearly identifiable as not corresponding to addition of a nucleotide to first polynucleotide 140. For example, labels that interact as a result of diffusion or due to a polymerase-directed nucleotide incorporation may have identical interaction lifetimes (statistically speaking). The lifetime is determined by the off rate of the interaction. The off rate is a constant that is governed by the nature of the interaction, temperature, salinity, buffer, and other factors. What distinguishes a true signal from a diffusive one is the percentage of time that the label is bound, and that is determined by the on rate. The on rate increases with the concentration of the label (in contrast to the off rate). For example, concentration corresponds to the probability of finding a molecule in a given volume. The local concentration of the label can be orders of magnitude higher for bound nucleotides compared with diffusive ones, because the nucleotide is held in the active site. Thus, the on-rate is much higher. While the labels may disassociate equally fast in the diffusive and specific states, the specific state results in the labels reassociating very rapidly. After the nucleotide is incorporated, the linker between the label and the nucleotide is severed. As a result, the next time the label interacts with the bridge, it has the same probability of floating away as the diffusive label.

In comparison, FIG. 1B illustrates a time at which polymerase 105 is adding nucleotide 121 (illustratively, G) to first polynucleotide 140 using at least the sequence of second polynucleotide 150 (e.g., so as to be complementary to a C in that sequence). Because polymerase 105 is acting upon nucleotide 121 to which label 131 is coupled (in some examples via linker 137), such action maintains label 131 at a location that is sufficiently close to bridge 110 for a sufficient amount of time to bring label 131 into contact with exposed region 113, so as to cause a sufficiently long change in an electrical characteristic, such as electrical conductivity or impedance, of bridge 110 as to be detectable using detection circuitry 160, allowing identification of nucleotide 121 as being added to first polynucleotide 140. Additionally, label 131 may have a property that, when contacting exposed region 113, imparts bridge 110 with an electrical characteristic, such as electrical conductivity or impedance, via which detection circuitry 160 may uniquely identify the added nucleotide as 121 (illustratively G) as compared to any of the other nucleotides.

Similarly, label 132 may have a property that, when contacting exposed region 113, alters an electrical characteristic, such as electrical conductivity or impedance, of bridge 110 via which detection circuitry 160 may uniquely identify the added nucleotide as 122 (illustratively T) as compared to any of the other nucleotides. Similarly, label 133 may have a property that, when contacting exposed region 113, alters an electrical characteristic, such as electrical conductivity or impedance, of bridge 110 via which detection circuitry 160 may uniquely identify the added nucleotide as 123 (illustratively C) as compared to any of the other nucleotides. Similarly, label 134 may have a property that, when contacting exposed region 113, alters an electrical characteristic, such as electrical conductivity or impedance, of bridge 110 via which detection circuitry 160 may uniquely identify the added nucleotide as 124 (illustratively C) as compared to any of the other nucleotides. It should be appreciated that labels 131, 132, 133, and 134 may have any suitable respective properties based upon which the electrical signal between first electrode 102 and second electrode 103 may vary in such a manner that detection circuitry 160 may identify nucleotides 121, 122, 123, 124 respectively coupled to those labels.

For example, FIGS. 2A-2C schematically illustrate examples of nucleotides with labels for use with a bridge that includes a polymer with metal-coated regions and an exposed region. In the nonlimiting example illustrated in FIG. 2A, label 231 includes a material of a first length (suggested by the rectangle having a particular length) that may contact exposed region 113 in such a manner as to change an electrical signal through bridge 110. Each of labels 232, 233, and 234 similarly includes a different length of material (not specifically labeled, but indicated by rectangles having different lengths than one another). Such variation in the labels' lengths provides different and distinguishable signals, e.g., currents or voltages, through bridge 110 using which the corresponding nucleotides may be identified.

In the nonlimiting example illustrated in FIG. 2B, label 231′ includes a sequence of two or more signal monomers (suggested by circles having different fills than one another) that respectively interact with (e.g., hybridize with) selected monomers within exposed region 113 in such a manner as to alter an electrical characteristic of bridge 110. The signal monomers of label 231′ may be located at any suitable location within the label. Each of labels 232′, 233′, and 234′ similarly includes two or more signal monomers (not specifically labeled, but indicated by circles having different fills than one another), although the particular types and sequences of those monomers vary between labels as intended to be suggested by the different fills of the circles indicating the monomers. Such variation in the labels' signal monomer types and sequences, when those monomers interact with (e.g., hybridize with) exposed region 113, provides different and distinguishable electrical signals, e.g., currents or voltages, through bridge 110 based upon which the corresponding nucleotides may be identified.

In one nonlimiting example, labels 231′, 232′, 233′, 234′ include respective oligonucleotides having at least partially different sequences than one another. The labels' respective oligonucleotide sequences may hybridize differently than one another with bridge 110 within exposed region 113. For example, signal monomers of label 231′ (suggested by circles having different fills than one another) may be nucleotides that are the same as or different from one another. The signal monomers in the other labels may be nucleotides that are different in sequence or in type, or both, from the first and second signal monomers of the other labels, such that each label 231′, 232′, 233′, 234′ has a unique sequence of first and signal monomers. The respective hybridization between the first and second signal monomers for each label and exposed region 113 may provide a particular electrical current or impedance through bridge 110. For example, label 231′ may have a sequence with a particular pair of bases that hybridizes with bases in exposed region 113 so as to modulate the electrical conductivity or impedance of bridge 110 to a first level; label 232′ may have a sequence with a particular pair of bases that hybridizes with bases in exposed region 113 so as to modulate the electrical conductivity or impedance of bridge 110 to a second level that is different from the first level; label 233′ may have a sequence with a particular pair of bases that hybridizes with bases in exposed region 113 so as to modulate the electrical conductivity or impedance of bridge 110 to a third level that is different from the first and second levels; and label 234′ may have a sequence with a particular pair of bases that hybridizes with bases in exposed region 113 so as to modulate the electrical conductivity or impedance of bridge 110 to a fourth level that is different from the first, second, and levels.

Similarly, labels 231′, 232′, 233′, and 234′ respectively may include any suitable combination, number, order, and type of monomer units (e.g., nucleotides) to allow electrical signals from different labels to be detected and distinguished from one another. For example, in FIG. 2C labels 231″, 232″, 233″, and 234″ may have different lengths than one another, e.g., may include any suitable number of monomers that may interact with (e.g., hybridize with) bridge 110 within exposed region 113. For example, the labels may include any suitable number of monomers (e.g., nucleotides), e.g., one, two, three, four, five, six, seven, eight, nine, ten, or more than ten monomers. It should be understood that labels 231″, 232″, 233″, and 234″ in some examples also may have different sequences than one another, in a manner such as described with reference to FIG. 2B. Additionally, such labels may alter any suitable electrical characteristic or characteristics of polymer chain 111. Additionally, such labels may be used with bridges that include any suitable number of polymer chains, e.g., bridges that include exactly two polymer chains, bridges that include a single polymer chain, or bridges that include more than two polymer chains.

FIG. 3 schematically illustrates an example composition for sequencing including a bridge that includes a polymer with metal-coated regions and an exposed region. In the example shown in FIG. 3, composition 300 may be similar to composition 100 described with reference to FIGS. 1A-1B, e.g., includes a substrate (not specifically shown), first electrode 302, second electrode 303, a polymerase (not specifically shown), bridge 310 including polynucleotide chain(s) 311 having first metal-coated region 312, second metal-coated region 312′, and exposed region 313, and nucleotides coupled to labels (not specifically shown). The polymerase may add nucleotides to a first polynucleotide using at least the sequence of a second polynucleotide, e.g., in a manner such as described with reference to FIGS. 1A-1B. Composition 300 may include other components such as described with reference to FIGS. 1A-1B, omitted here.

In the example illustrated in FIG. 3, polynucleotide chain(s) 311 may include first and second polynucleotide chains, e.g., dsDNA, coupled to first and second electrodes 302, 303 via the metal of first and second metal-coated regions 312, 312′. The labels coupled to the nucleotides may have sequences that respectively alter hybridization between the polynucleotide strands within exposed region 313, providing distinguishable electrical signal through bridge 310. In some examples, the labels may include modified nucleotides, such as nucleotides with modified backbones (e.g., phosphorothioate DNA), modified sugars (e.g., 2′ o-methyl or 2′ OH (RNA)), modified bases (e.g., methylated bases), or nucleic acid analogs such as peptide-nucleic acids (PNA) or locked nucleic acids (LNA). Such labels, when contacted with exposed region 313, may alter hybridization between the polynucleotide chains in such a manner as to detectably change the flow of current or impedance through bridge 310. Modified nucleotides may alter the manner in which the polynucleotide chains hybridize with one another. For instance, bulky base modifications in labels may alter the geometry between the polynucleotide chains, thus affecting electrical conduction characteristics. By similar mechanisms, modifications to the sugar or backbone may have similar effects.

In other examples, the labels may include respective DNA-binding proteins. Such labels, when used with polynucleotide chain(s) (such as DNA, or enantiomeric DNA) may alter hybridization between, or the electrical conduction characteristics of, the polynucleotide chains in such a manner as to detectably change the flow of current or impedance through a bridge including those polynucleotide chains. Non-limiting examples of DNA-binding proteins that may be used in the present labels include molecular sleds, transcription factors, proteins that function as the binding domain of transcription factors such as designer zinc finger and leucine zippers, catalytically inactive nucleases (e.g,. Hind III, Eco RI), histones, RecA (and other recombinases), and catalytically inactive Crispr-Cas9 and analogs thereof.

In still other examples, labels may include respective intercalators, such as minor groove binders (MGBs), DNA intercalators, or peptide intercalators. Nonlimiting examples of MGBs include distamycin, netropsin, bisbenzimadazoles, bisamidines, mithramycin, and chromomycin, and their analogs and derivatives. DNA intercalators may include molecules with planar aromatic or heteroaromatic groups capable of stacking between adjacent DNA base pairs. Examples of DNA intercalators that may be used in the present labels include daunomycin, doxorubicin, epirubicin, dactinomycin, ditercalinium, bleomycin, elsamicin A, m-AMSA, mitoxantrone, acridines, and ethidium bromide. For example, ethidium bromide is believed to lengthen the DNA helix, thus altering the electrical conductivity of the DNA helix. Peptide based DNA intercalators may include peptide backbones.

In some examples, labels may include respective intertwining alpha helices. Such alpha helix-based labels, when used with double-stranded polymer bridges (e.g., DNA), may alter hybridization between double-stranded chains in such a manner as to detectably change the flow of current or impedance through the bridge. Examples of alpha helices that may be used in the present labels include peptide coiled coils and leucine zippers, such as described in greater detail elsewhere herein.

In some examples, bridge 110 described with reference to FIGS. 1A-1B may include any suitable number of polypeptide chains, e.g., one or more, two or more, or three or more polypeptide chains, and the labels coupled to the nucleotides may include respective proteins, peptides, or intercalators that alter an electrical characteristic of one or both of the first and second polypeptides. For example, one or more of the polypeptide chains of bridge 110, and in some examples each of the polypeptide chains of bridge 110, may directly contribute to electron transfer between first electrode 102 and second electrode 103. Without wishing to be bound by any theory, it is believed that such electron transfer may be enabled using, e.g., pi-stacking of aromatic amino acid side chains (such as those of tyrosine, tryptophan, or phenylalanine) in each of the chains. However, other transport mechanisms besides pi-stacking may be used, alone or in combination with pi-stacking. The labels respectively may confer changes in electrical conductivity (an example electrical characteristic) to one or more of the polypeptide chains of bridge 110, for example using formation of a complex such as a dimer, trimer, or higher mer. As such, each of the labels and one or more of the polypeptide chains of bridge 110 may in some examples work together to transfer electrons from first electrode 102 to second electrode 103. The labels may contact the exposed region 113 in such a manner as to alter the electrical conductivity of the resulting label-polypeptide chain complex differently than one another, thereby providing different electrical signals via which nucleotides may be identified.

In other examples, bridge 110 described with reference to FIGS. 1A-1B may include a single-stranded polymer chain, and the labels coupled to the nucleotides respectively may contact exposed region 113 in such a manner as to modulate the electrical conductivity or impedance of bridge 110, based upon which modulation the identity of the corresponding nucleotides may be determined. For example, polymer chain 111 may be or include a conjugated polymer including a first delocalized set of orbitals, and the labels each may include a respective delocalized set of orbitals that, when associated with the conjugated polymer in exposed region 113, share electrons with the first delocalized set of orbitals in such a manner as to alter electrical conductivity of the conjugated polymer. Illustratively, single-stranded conjugated polymer chain 111 and each of the labels may form a corresponding charge transfer complex. For example, the conjugated polymer chain 111 may act as a donor and the labels may act as respective acceptors. Each label and the conjugated polymer chain 111 may form a donor:acceptor complex via π-π interactions, via which electrons may transfer from the donor to the acceptor so as to cause a change in electrical conductivity of the conjugated polymer chain. The extent of electron transfer from a donor to an acceptor depends on the extent of overlap between the molecular orbitals of the donor with the molecular orbitals of the acceptor. As such, labels which include different molecular orbitals may be expected to have different π-π interactions with the conjugated polymer chain, and as such to detectably change the electrical conductivity of the conjugated polymer chain differently, thus permitting detection circuitry 160 to uniquely identify the nucleotides to which such labels are coupled.

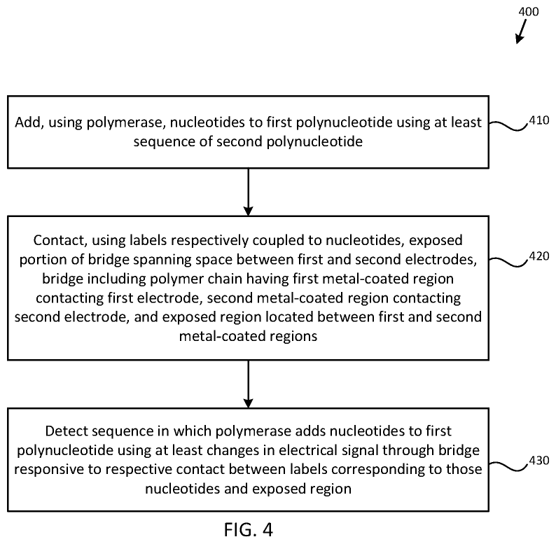

It will be appreciated that compositions such as described with reference to FIGS. 1A-1B, 2A-2C, and 3 suitably may be used for sequencing polynucleotides. For example, FIG. 4 illustrates an example flow of operations in a method for sequencing using a bridge that includes a polymer with metal-coated regions and an exposed region, and nucleotide labels that contact the exposed region to alter an electrical characteristic of the bridge. Bridges 110 and 310 are nonlimiting examples of such a bridge.

Method 400 illustrated in FIG. 4 may include adding, using a polymerase, nucleotides to a first polynucleotide using at least a sequence of a second polynucleotide (operation 410). For example, polymerase 105 described with reference to FIGS. 1A-1B may add nucleotides 121, 122, 123, 124 to first polynucleotide 140 using at least the sequence of second polynucleotide 150.

Method 400 illustrated in FIG. 4 also may include contacting, using labels respectively coupled to the nucleotides, an exposed region of a bridge spanning a space between first and second electrodes (operation 420). The bridge may include a polymer chain having a first metal-coated region contacting the first electrode, a second metal-coated region contacting the second electrode, and an exposed region located between the first and second metal-coated regions. For example, bridge 110 may include polymer chain 111 having first metal-coated region 112 contacting first electrode 102, second metal-coated region 112′ contacting second electrode 103, and exposed region 113 located between the first and second metal-coated regions. In nonlimiting examples such as described with reference to FIG. 3, the polymer chain may include polynucleotide, such as DNA. In various examples, the polynucleotide may be single-stranded, or may be double-stranded.

Method 400 illustrated in FIG. 4 also may include detecting a sequence in which the polymerase adds the nucleotides to the first polynucleotide using at least changes in electrical signal through the bridge that are responsive to respective contact between the labels corresponding to those nucleotides and the exposed region (operation 430). For example, labels 131, 132, 133, 134 respectively may contact exposed region 113 in a manner such as described with reference to FIG. 1B. Detection circuitry 160 may detect changes in electrical signal through bridge 110 resulting from such contact, and nucleotides 121, 122, 123, 124 respectively may be detected using such changes.

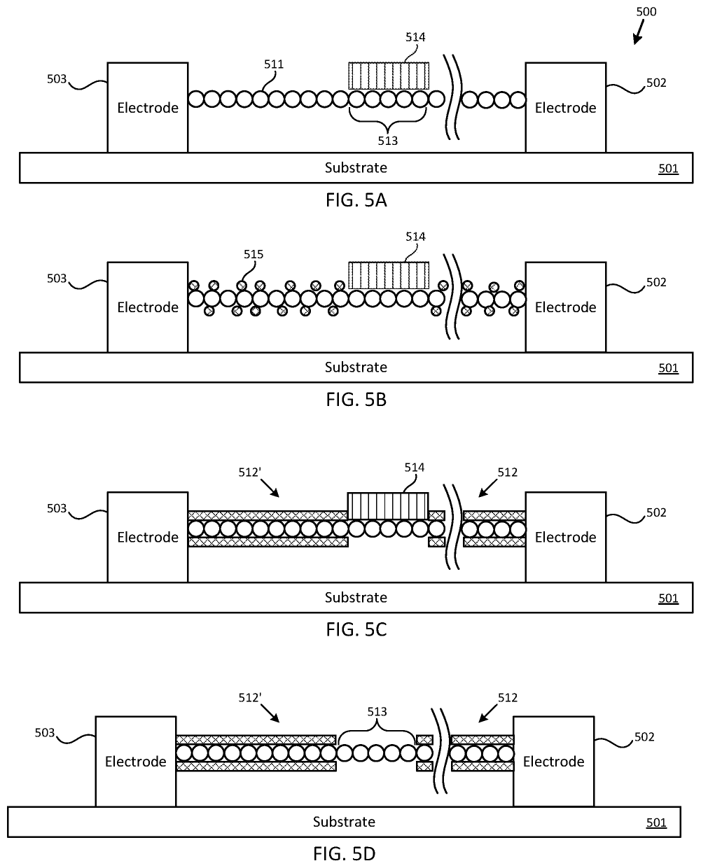

It will be appreciated that bridges such as described herein may be prepared using any suitable order and combination of operations. For example, FIGS. 5A-5D schematically illustrate an example flow of operations in a method for making a bridge that includes a polymer with metal-coated regions and an exposed region. In the nonlimiting example illustrated in FIG. 5A, a first end of polymer 511 is coupled to first electrode 502, and a second end of polymer 511 is coupled to second electrode 503. Such couplings may be via any suitable bond(s). Mask 514 may be coupled to region 513 of polymer 511 which it is desired not to coat with metal. Illustratively, mask 514 may include any suitable molecular or biomolecular entity that can be associated to the bridge via directed intermolecular interactions, and which inhibits metallization at the masked location of the bridge. Mask 514 may be coupled to polymer chain 511 using any suitable operation(s), such as oligonucleotide hybridization, oligonucleotide strand invasion, directed pi-pi interactions, metal-ligand interactions, and the like. In one nonlimiting example in which polymer chain 511 includes a single-stranded or double-stranded polynucleotide, mask 514 may include a site-selective protein, such as a RecA protein or D-site-binding protein, that selectively couples to the sequence of the polynucleotide in region 513 in such a manner as to inhibit metal from coating polymer chain 511 in that region. In another example certain sections of the polymer itself may be designed in such a way that they may be metal coated, or may be metal coated at a much slower rate than other sections. For example, if metallization utilizes the initial seeding of clusters from a second metal, a section of the polymer may be designed in such a way that no, or substantially no, seeding occurs in this section.

For example, as illustrated in FIG. 5B, metal seeds 515 may be coupled to polymer chain 511 at regions other than where mask 514 is coupled to the polymer chain. For example, polymer chain 511 may be contacted with a first solution including metal ions, and the metal ions may become coupled to polymer chain 511 except at mask 514 to provide metal seeds. The metal seeds 515 may provide nucleation sites for an electroless plating operation. For example, polymer chain 511 may be contacted with a second solution including metal ions and a reducing agent that reduces the metal ions in such a manner as to generate first and second metal-coated regions 512, 512′ in a manner such as illustrated in FIG. 5C. The metal of metal-coated region 512 may be coupled to (e.g., may at least partially cover) first electrode 502, and the metal of metal-coated region 512′ be coupled to (e.g., may at least partially cover) second electrode 503. It will be appreciated that the metals used in the first and second solutions may be, but need not necessarily be, the same as one another. For example, the first solution may include a salt of silver, platinum, palladium, gold, copper, nickel, mercury, lead, calcium, manganese, lanthanum or other lanthanides, cobalt, iron, zinc, or rhodium, or a combination thereof, and the second solution may include the same or a different salt of silver, platinum, palladium, gold, copper, nickel, cobalt, zinc, chromium, iron, vanadium, or rhodium, or a combination thereof. Illustratively, the first solution may include silver ions forming silver seeds 515, and the second solution may include gold ions that plate using the silver seeds as nucleation sites, such that the metal of metal-coated regions 512, 512′ includes both silver and gold. Following formation of metal-coated regions 512, 512′, mask 514 may be removed via any suitable method (such as increased temperature, change in pH, change in buffer, change in salt concentration, electrical pulse, addition of a suitable enzyme, and the like) such as illustrated in FIG. 5D. Alternatively, mask 514 may be removed by any suitable method after formation of metal seeds 515, or may be left in place without removal, or may not necessarily be used at all. For example, even in the absence of mask 514, electroless plating may not coat region 513 with metal because metal seeds 515 substantially are absent from region 513.

In some examples in which polymer chain 511 includes a single-stranded or double-stranded polynucleotide, metal seeds 515 may be form selectively in regions outside of region 513 using the sequence of the polynucleotide(s). For example, metal ions may have a relatively high affinity for C and G nucleotides (e.g., for amine groups) as compared to their affinity for A and T (or U) nucleotides or abasic nucleotides. As such, by providing a relatively high concentration of A and T nucleotides—or even abasic nucleotides—in region 513 of polymer chain 511 and a relatively low concentration of C and G nucleotides outside of region 512, polymer chain 511 may be selectively coupled to metal seeds 515 at regions outside of region 513 even without the use of mask 514.

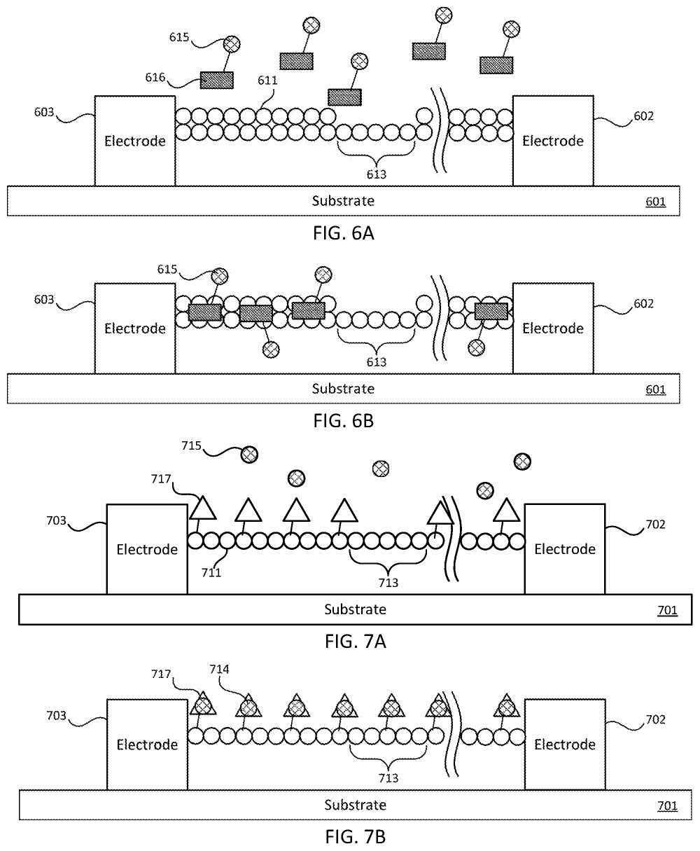

FIGS. 6A-6B schematically illustrate another example flow of operations in a method for making a bridge that includes a polymer with metal-coated regions and an exposed region. In the example shown in FIG. 6A, polymer chain 611 is single-stranded in region 613, and double-stranded outside of region 613. Illustratively, polymer chain 611 may include ssDNA in region 613, and dsDNA outside of region 613. In some examples, polymer chain 611 may be prepared by coupling a fully double-stranded polymer to electrodes 602, 603, and then cutting away a portion of one of the strands to form single-stranded region 613, e.g., using a cutting enzyme. Polymer chain 611 may be contacted with a solution including intercalator 616 coupled to metal seeds 615. As illustrated in FIG. 6B, the intercalator selectively may become coupled to (e.g., may intercalate into) double-stranded regions as compared to single-stranded region 613, and thus may selectively couple metal seeds to double-stranded regions as compared to single-stranded region. The metal seeds 615 then may be used in a plating process such as described with reference to FIG. 5C.

FIGS. 7A-7B schematically illustrate another example flow of operations in a method for making a bridge that includes a polymer with metal-coated regions and an exposed region. In the example shown in FIG. 7A, polymer chain 711 (e.g., a conjugated polymer or polypeptide) includes functional groups 717 in regions outside of region 713, and functional groups 717 are substantially absent in region 713. Polymer chain 711 may be contacted with a solution including metal seeds 715. As illustrated in FIG. 7B, the metal seeds 715 selectively may become coupled to functional groups 717 in regions outside of region 713. The metal seeds 715 then may be used in a plating process such as described with reference to FIG. 6C.

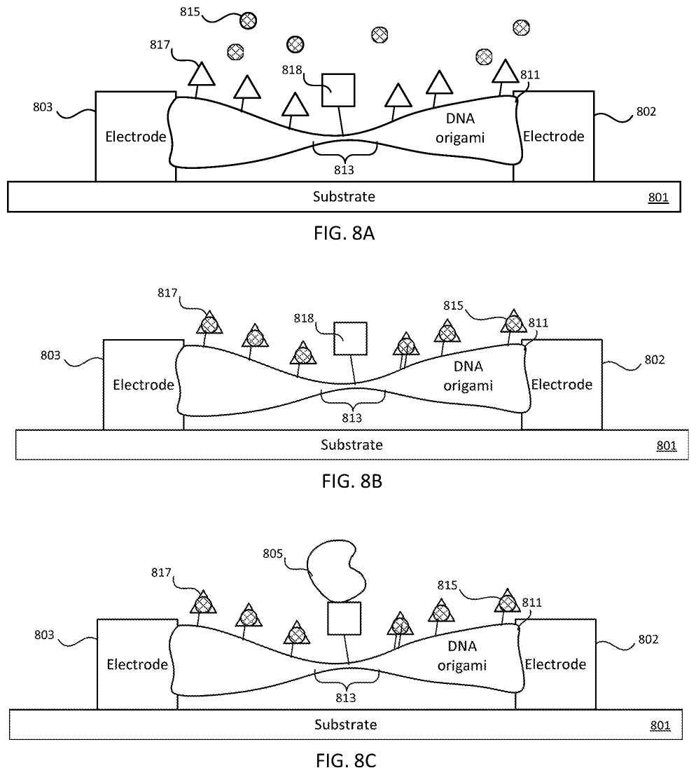

FIGS. 8A-8C schematically illustrate another example flow of operations in a method for making a bridge that includes a polymer with metal-coated regions and an exposed region. In the example shown in FIG. 8A, polymer chain 811 (here, DNA origami) includes functional groups 817 in regions outside of region 813, and functional groups 817 are substantially absent in region 813. Additionally, functional group 818 may be provided within region 813 for use in coupling a polymerase to DNA origami 811. For example, oligonucleotide staples that are functionalized with respective functional groups may be used to couple functional groups 817, 818 to selected regions of DNA origami 811. Illustratively, functional groups 817 may include oligonucleotides that extend outwardly from the DNA origami. DNA origami 811 may be contacted with a solution including metal seeds 815. As illustrated in FIG. 8B, the metal seeds 815 selectively may become coupled to functional groups 817 in regions outside of region 813. Illustratively, in examples where functional groups 817 include oligonucleotides, the metal seeds 815 may couple to the oligonucleotides. The metal seeds 815 then may be used in a plating process such as described with reference to FIG. 6C. As illustrated in FIG. 8C, polymerase 805 may be coupled to functional group 818 for use in sequencing a polynucleotide in a manner such as described with reference to FIGS. 1A-1B.

In examples such as described with reference to FIGS. 5A-5D, 6A-6B, 7A-7B, and 8A-8C, the polymer chain may be coupled to the first and second electrodes in any suitable manner. For example, polymer chains including polynucleotides such as ssDNA or dsDNA may be stretched across the first and second electrodes using any suitable combination of molecular combing, electrophoretic stretching, and hydrodynamic stretching.

Additionally, in examples such as described with reference to FIGS. 5A-5D, 6A-6B, 7A-7B, and 8A-8C, the metal seeds may have any suitable size. For example, metal seeds that form out of a metal salt solution onto a polymer chain (e.g., such as described with reference to FIGS. 5A-5D) may have diameters from about 0.5 nm to about 50 nm, e.g., from about 1 nm to about 20 nm, e.g., from about 1 nm to about 10 nm. Metal seeds that are coupled to intercalators that intercalate into double-stranded polymer chains, or are coupled to the polymer chain via functional groups, may have diameters from about 0.5 nm to about 50 nm, e.g., from about 1 nm to about 20 nm, e.g., from about 1 nm to about 10 nm.

### WORKING EXAMPLES

Additional examples are disclosed in further detail in the following examples, which are not in any way intended to limit the scope of the claims.

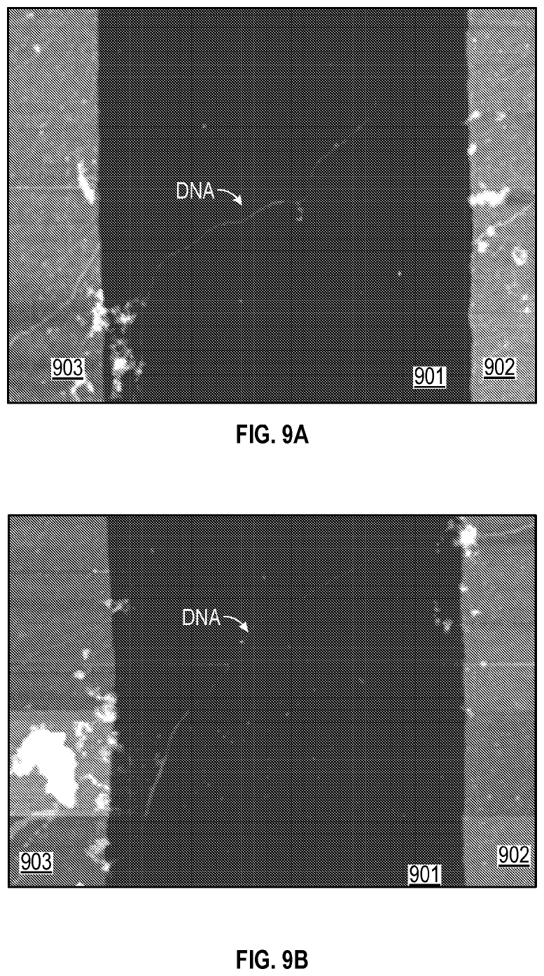

FIGS. 9A-9B are atomic force microscopy (AFM) images of individual DNA duplexes combed across a space between electrodes, according to one example. This sample was prepared by combing a solution of λ-DNA across the surface of a substrate 901 including gold electrodes 902, 903 separated by ˜3 μm. The solution was combed by pulling a droplet across the surface using a pipette tip. This example demonstrates that DNA may be coupled to electrodes, e.g., for use in a bridge.

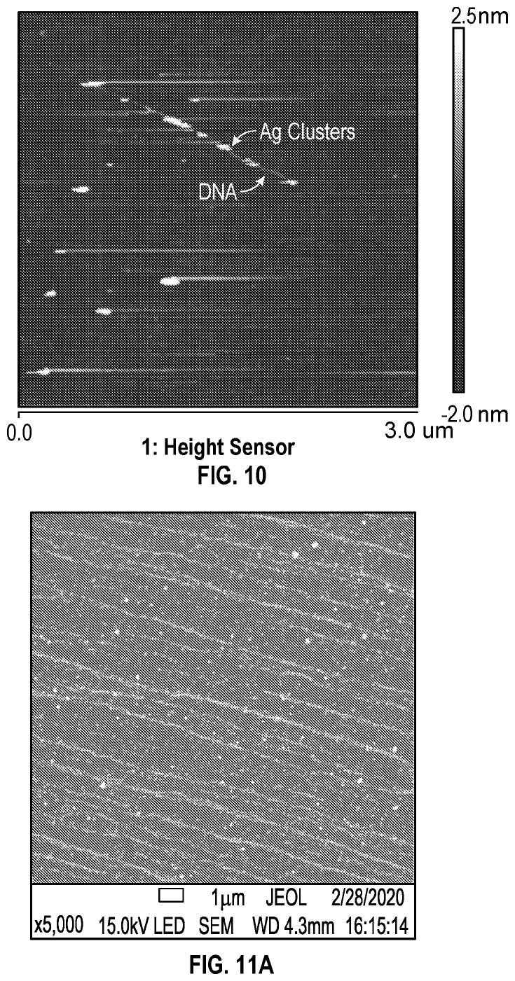

FIG. 10 is an AFM image of DNA after seeding with silver ions and reducing to silver nanoparticles, according to one example. This sample was prepared by combing DNA over a hydrophobic surface, incubating with a solution of AgNO₃, and then reducing the silver ions with a solution of hydroquinone. This example demonstrates that DNA may be seeded with metal, e.g., for use in a bridge.

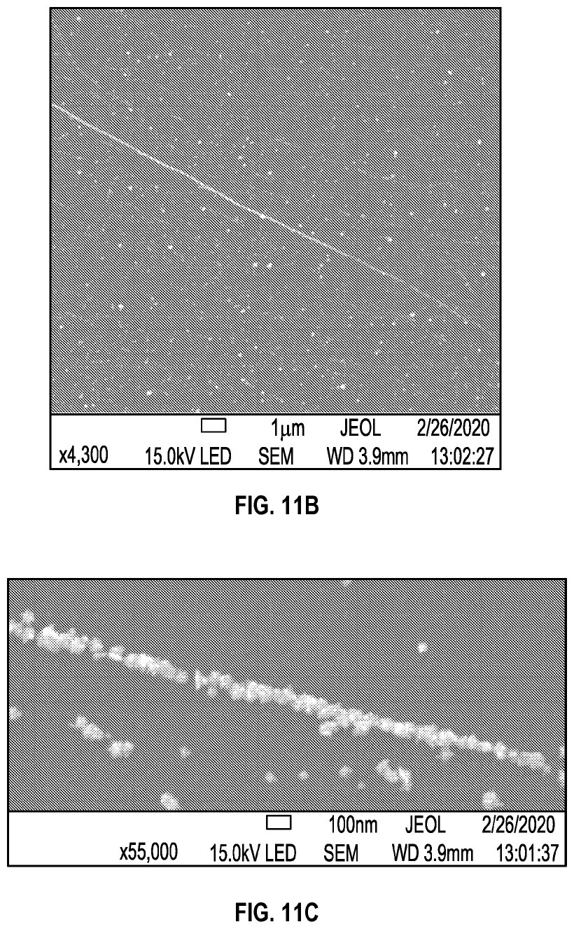

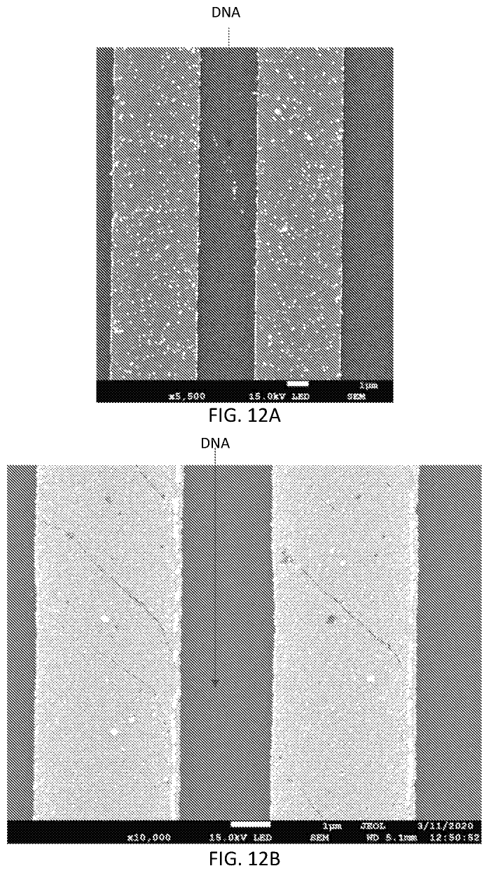

FIGS. 11A-11C are scanning electron microscopy (SEM) images of metalized DNA that has undergone seeding with silver clusters and then electroless plating with gold, according to one example. This sample was prepared by combing DNA over a hydrophobic surface, incubating with a solution of AgNO₃, reducing the silver ions with a solution of hydroquinone, then carrying out electroless plating of Au by exposing the surface to KAu(SCN)₄ in the presence of hydroquinone. This example demonstrates that DNA may be coated with metal using a process including seeding followed by plating, e.g., for use in a bridge.

FIGS. 12A-12B are SEM images of DNA that was combed across a space between electrodes, seeded with silver clusters, and then electroless plated with gold, according to one example. This sample was prepared by combing DNA over a hydrophobic surface, incubating with a solution of AgNO₃, reducing the silver ions with a solution of hydroquinone, then carrying out electroless plating of Au by exposing the surface to KAu(SCN)₄ in the presence of hydroquinone. This example demonstrates that DNA may be coupled to electrodes and coated with metal using a process including seeding followed by plating, e.g., for use in a bridge.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A composition, comprising:
first and second electrodes separated from one another by a space;
a bridge spanning the space between the first and second electrodes, the bridge comprising a polymer chain having a first metal-coated region contacting the first electrode, a second metal-coated region contacting the second electrode, and an exposed region located between the first and second regions;
first and second polynucleotides;
a plurality of nucleotides, each nucleotide coupled to a corresponding label;
a polymerase to add nucleotides of the plurality of nucleotides to the first polynucleotide using at least a sequence of the second polynucleotide; and
detection circuitry to detect a sequence in which the polymerase adds the nucleotides to the first polynucleotide using at least changes in an electrical signal through the bridge, the changes being responsive to contact between the labels corresponding to those nucleotides and the exposed region.

2. The composition of claim 1, wherein the polymer chain comprises a polynucleotide.

3. The composition of claim 2, wherein the polynucleotide comprises DNA.

4. The composition of claim 2, wherein the polynucleotide is single-stranded.

5. The composition of claim 2, wherein the polynucleotide is double-stranded.

6. The composition of claim 1, wherein the first and second metal-coated regions comprise metal nanoparticles coupled to the polymer chain.

7. The composition of claim 1, wherein the first and second metal-coated regions comprise metal plated on the polymer chain.

8. The composition of claim 1, wherein the metal is selected from the group consisting of: silver, platinum, palladium, gold, copper, nickel, cobalt, zinc, and rhodium.

9. A method, comprising:
adding, using a polymerase, nucleotides to a first polynucleotide using at least a sequence of a second polynucleotide;
contacting, using labels respectively coupled to the nucleotides, an exposed region of a bridge spanning a space between first and second electrodes, the bridge comprising a polymer chain having a first metal-coated region contacting the first electrode, a second metal-coated region contacting the second electrode, and the exposed region located between the first and second metal-coated regions; and
detecting a sequence in which the polymerase adds the nucleotides to the first polynucleotide using at least changes in electrical signal through the bridge that are responsive to respective contact between the labels corresponding to those nucleotides and the exposed region.

10. The method of claim 9, wherein the polymer chain comprises a polynucleotide.

11. The method of claim 10, wherein the polynucleotide comprises DNA.

12. The method of claim 10, wherein the polynucleotide is single-stranded.

13. The method of claim 10, wherein the polynucleotide is double-stranded.

14. The method of claim 9, wherein the first and second metal-coated regions comprise metal nanoparticles coupled to the polymer chain.

15. The method of claim 9, wherein the first and second metal-coated regions comprise metal plated on the polymer chain.

16. The method of claim 9, wherein the metal is selected from the group consisting of: silver, platinum, palladium, gold, copper, nickel, cobalt, zinc, and rhodium.
